(19) United States
(12) Patent Application Publication
(10) Pub. No.: US 2006/0079963 A1
(43) Pub. Date: Apr. 13, 2006

(54) Semiconstrained shoulder prosthetic for treatment of rotator cuff arthropathy

(76) Inventor: Regan Hansen, Clarkston, WA (US)

Correspondence Address: PEDERSEN & COMPANY, PLLC, P.O. BOX 2666, BOISE, ID, 83701, US

(21) Appl. No.: 10/962,052

(22) Filed: Oct. 7, 2004

(51) Int. Cl. A61F 2/40; A61B 17/90; A61B 17/17

(52) U.S. Cl. 623/19.11; 623/19.13; 606/87

(57) ABSTRACT

A glenoid component for treatment of rotator cuff arthropathy includes attachment to the coracoid process by a pin or post imbedded into a hole formed in the coracoid process. The glenoid component preferably also has a keel for extending into the glenoid fossa and protrusions such as ridges cemented to the acromion process. This way, an attachment point is preferably provided in/on the coracoid process, the acromion process, and the glenoid fossa, and at least two of the attachment points include a protrusion extending into a hole/slot drilled or otherwise formed in the bone. A jig is used for guiding/drilling into the bone, wherein the jig has both a glenoid fossa drill guide and a coracoid drill guide. The coracoid drill guide includes structure that abuts against opposing sides of the base of the coracoid process to prevent rotation of the jig relative to the scapula.

### BACKGROUND OF THE INVENTION

1. Field of the Invention

The present invention relates to a shoulder replacement device for treatment of rotator cuff arthropathy. More specifically, the present invention relates to shoulder replacement devices comprising a glenoid component and a humeral component. The invention further relates to a glenoid component having multiple points of attachment to the scapula for increased stability.

2. Related Art

The gleno-humeral joint or shoulder joint is the most freely moveable joint in the body. At the gleno-humeral joint, the head of the humerus articulates with the glenoid fossa of the scapula. A band of fibrocartilage passes around the rim of the joint, reducing the friction between the articulating surfaces. The shoulder joint is protected from above by an arch formed by the acromion process and coracoid process of the scapula, and by the clavicle. Most of the stability of the joint is provided by the joint capsule, the ligaments, the bicep tendons, and the tendons of the subscapularis, supraspinatus, infraspinatus, and teres minor muscles, which together form the musculotendinous or rotator cuff.

Rotator cuff arthropathy develops as a result of tears to either the soft tissues or the tendons of the shoulder. These tears are classified as acute tears or chronic tears. In an acute tear, the rotator cuff ruptures after a specific traumatic event to the shoulder without a preceding history of shoulder problems. Acute tears are often massive, involving ¾ of the rotator cuff tendons, and in turn cause chronic migration of the humeral head in the superior direction. Chronic tears are more common than acute tears. Chronic tears are micro tears, or degenerative tears that occur over years. As a result of both acute and chronic tears, severe arthritis develops in the patient's shoulder due to the incongruity and instability of the shoulder. Further, tears in the rotator cuff and the loss of cartilage in the joint cause significant translation or hypertranslation of the humeral head in the superior direction. This superior translation forces the humeral head to abut against the inferior surface (subacromial space) of the acromion process and coraco-acromial ligament causing erosion of the acromion process, the humeral head, and the glenoid fossa, as well as severe pain to the patient.

Deterioration of the supporting cartilage and bone results in pain and instability of the shoulder joint and possibly subsequent dislocations that often necessitate the implantation of a prosthetic shoulder joint or fusion to stabilize the shoulder joint. Because the stability of the shoulder joint depends upon the muscles and tendons surrounding the joint, patients with rotator cuff arthropathy lack the muscle tissue and the attachments to prevent superior migration of the humeral head. One of the goals of orthopedic surgeons developing shoulder prosthetics is to develop a shoulder prosthetic that increases the stability of the joint without limiting the range of motion.

During the development of shoulder prosthetics, three primary considerations have emerged. The first consideration being that the glenoid fossa is vertically oriented and has a small surface area. This small surface area has made it difficult to securely fit an implant solely to the glenoid fossa, and in turn these implants have failed by loosening at the glenoid interface. The second consideration is that rotator cuff arthropathy by definition is always severely affected by the existent disease causing gleno-humeral joint destruction and proximal humeral head migration, therefore, the prosthetic design should provide for stability greater than present in the normal shoulder articular surfaces. The third consideration is that the range of motion in the shoulder is very great; a standard, single ball-in-socket design may not suffice because it contains the humeral head completely within the glenoid component and maintains a fixed point of rotation.

In response to the development of a variety of prosthetic systems, the systems have been categorized as: anatomical or unconstrained, semiconstrained (having a hooded glenoid component), or constrained (a ball-in-socket unit). Almost all of these designs include a concave glenoid component and a cooperating, generally spherical humeral head to replace the stabilizing functions of the rotator cuff. The designs are generally categorized according to the extent of “capture” of the humeral head in the glenoid component, that is, the extent to which the glenoid component extends around/surrounds the humeral head. Many of these designs have included extensive attachments of the glenoid component to the scapula by stems, wedges, screws and bolted flanges, however, many of these attachments are implanted entirely within the glenoid fossa.

Referring to the above categories, anatomical or “unconstrained” designs have been designed in order to emulate the normal articulation surface of the gleno-humeral joint. These designs feature little if any “capturing” of the humeral head.

Constrained designs feature glenoid components that extend around the humeral head to an extent that warrants the name “ball-in-socket”. These designs afford increased stability via this capture of the humeral head, but, in turn, they severely limit the patient's range of motion.

In order to create a compromise between the unconstrained units and the constrained ball-in-socket units, a hood was placed upon the glenoid component of an unconstrained unit to extend the articulation surface, creating a semiconstrained design. These designs provide increased stability, compared to unconstrained units, but greater range of motion than the ball-in-socket units.

In recent years, the semiconstrained system has been preferred over the constrained systems because it allows a greater range of motion. The semiconstrained total shoulder systems include the hooded glenoid component. The hood is designed to hold the humeral head in place against the prosthetic socket, preventing superior translation of the humeral head, and too a lesser degree, preventing anterior and posterior instability. However, in many semiconstrained systems, the glenoid component bone-cement junction loosens due to the force exerted on the on the single attachment point in the glenoid fossa. In response to the unstable hood component, one design extends the hood and has it rest against the acromion to offer additional support. However, there is still potential for the glenoid component to loosen. Therefore, the instant inventor believes there is still a need for a semiconstrained shoulder prosthetic that is well secured to the scapula.

Issued patents relating to gleno-humeral joint prosthetics are reviewed hereinafter.

Stroot (U.S. Pat. No. 3,979,778) discloses a shoulder prosthesis consisting of a humeral and a glenoid component, each of which has a spherical articular surface. The radius of curvature of the glenoid component is substantially greater than that of the humeral component, providing what might be called a wandering fulcrum. Stroot further discloses a glenoid component that is fixed to the bone with glue or cement at points of contact with the glenoid fossa, acromion process and coracoid process.

Dines et al. (U.S. Pat. No. 4,865,605) discloses a shoulder prosthesis wherein the humeral component has a modular design which enables different available sized heads to be placed onto a stem which has been implanted in the proximal humerus.

Maroney et al. (U.S. Pat. No. 6,620,197) discloses a prosthetic assembly for a shoulder including a stem component configured to be implanted into a medullary canal of a humerus of the patient. The assembly also includes a prosthetic head component configured to be secured to a proximal end portion of the stem component. The head component has a glenoid-bearing portion configured to bear against a glenoid surface, and an acromion-bearing portion, which is configured to bear against an acromion.

Wolf (U.S. Pat. No. 5,507,819) discloses a prosthetic glenoid for use in the shoulder comprising a cup having three flanges.

### SUMMARY OF THE INVENTION

The present invention relates generally to shoulder prosthetics, and, more specifically, to shoulder prosthetics comprising a glenoid component and a humeral component. The invented shoulder prosthetic is adapted to be securably attached to the scapula to prevent the glenoid component from loosening. Preferably, the glenoid component is constrained superiorly and semiconstrained inferiorly to allow more mobility in the gleno-humeral joint.

The preferred embodiment of the invented glenoid component is generally concave or cup-shaped and comprises structure for attaching the component to at least two, and preferably three, of the most lateral projections/extremities of the scapula, which are the acromion process, the coracoid process, and the glenoid fossa. The preferred glenoid component comprises at least two member(s) that is/are drilled or otherwise sunk into the bone. A third attachment area may be included that comprises member(s) anchored to the bone with cement and without being drilled or sunk into the bone. The preferred three areas of attachment greatly enhance the stability of the joint.

In the preferred embodiment, the glenoid component has an area of attachment on its inferior-medial surface, its anterior-medial surface, and its posterior-superior surface. Preferably, the inferior-medial attachment structure is a keel, a plurality of pegs, or other elongated extension or protrusion for attachment to the glenoid fossa. The attachment structure on the anterior-medial side of the glenoid component is preferably a peg, post, or other protrusion for securing the glenoid component to the base of the coracoid process. The attachment structure on the posterior-superior side of the glenoid component is preferably a plurality of ridges or other protrusions for use in cementing the glenoid component to the acromion process. These various attachment protrusions are preferably integral with the main body of the glenoid component, in that they are preferably molded with the main body as one unit. Less preferably, the various attachment protrusions may be non-integral with the main body, for example, rigidly attached to, connected, or extended through the main body rather than molded with the main body.

In the preferred embodiment, a lateral surface of the glenoid component comprises a concave interior curvature for articulating with the humeral component and a hood extension for preventing the humerus from translating in the superior direction. Preferably, the hood is extended anteriorly and posteriorly to provide stability in both the anterior and posterior positions.

The humeral component may comprise a generally spherical or hemi-spherical member that is anchored by a stem system that extends into the humerus. Alternatively, other humeral components and/or other systems for anchoring the humeral component to the humerus may be used.

In accordance with the preferred embodiment of the present invention, there is provided a preferred method of implanting the invented shoulder prosthetic. The preferred method of implantation may be generally according to the Cofield anterior deltopectoral extensile approach, or a modification thereof. The Cofield approach is well understood in the field (Matsen, F. A., III, M.D. & Rockwood, C. A., Jr., M.D. (Eds.). (1990) The Shoulder, Volume 2. Philadelphia, Pa.: W.B. Saunders Company.). An invented jig or template apparatus may be used to carry out the preferred method.

### BRIEF DESCRIPTION OF THE DRAWINGS

FIG. 1 is a lateral view of a left scapula with the glenoid slot and coracoid base slot shown.

FIG. 2 is a lateral view of the preferred prosthetic glenoid component oriented in a left scapula.

FIG. 3 is an anterior view of the preferred prosthetic glenoid component shown in combination with the preferred prosthetic humeral component.

FIG. 4 is an anterior cross-sectional view of the embodiment shown in FIG. 3.

FIG. 5 is an inferior view of the preferred humeral head component.

FIG. 6 is a cross-sectional view of the embodiment shown in FIG. 4.

FIG. 7 is a lateral view of the preferred prosthetic glenoid component.

FIG. 8 is a medial view of the preferred prosthetic glenoid component.

FIG. 9 is an anterior view of the embodiment shown in FIG. 8.

FIG. 10 is a posterior view of the embodiment shown in FIGS. 8 and 9.

FIG. 11 is an inferior view of the embodiment shown in FIGS. 8-10.

FIG. 12 is a lateral view of one embodiment of an invented jig used in preferred methods of rotator cuff arthropathy, the jig being shown in relationship to the bones of the left scapula.

FIG. 13 is an anterior view of the jig shown in FIG. 12.

FIG. 14 is a lateral view of the jig shown in FIGS. 12 and 13, wherein the jig is shown removed from the scapula.

FIG. 15 is a detail view of one embodiment of a coracoid drill hole guide of the jig shown in FIGS. 12-14, wherein the coracoid drill hole guide is viewed from a direction that reveals both prongs.

FIG. 16 is a detail view of one embodiment of a coracoid drill hole guide of the jig shown in FIGS. 12-15, wherein the coracoid drill hole guide is viewed from a direction that reveals a single prong.

FIG. 17 is an anterior view of the jig shown in FIGS. 12-17, illustrating to best advantage the coracoid drill hole guide and the jig handle.

### DETAILED DESCRIPTION OF THE INVENTION

Referring to the figures, there is shown one, but not the only, embodiment of the invented semiconstrained shoulder prosthetic. In this Description and the claims, the term “proximal” means toward the center/torso of the body, whereas the term “distal” indicates a point farthest from the center/torso of the body. Other directional terms of reference used herein are: “superior” meaning toward the head/top; “inferior” meaning away from the head or toward the bottom; “anterior” meaning toward the front; “posterior” meaning toward the back; “medial” meaning inwardly from the side toward the midline of the body; and, “lateral” meaning outwardly from the midline of the body toward the side.

The preferred embodiment of the invented semiconstrained shoulder prosthetic comprises a glenoid component 100 and a humeral component 200 for replacement of a patient's deteriorated gleno-humeral joint. Preferably, the glenoid component 100 shaped to offer the patient a greater range of motion. Further, the glenoid component 100 offers three points of attachment to provide greater stability to the joint. The humeral component 200 preferably comprises an elongated stem 210 and a semicircular head portion 220. Preferably, the humeral component 200 articulates with the glenoid component 100.

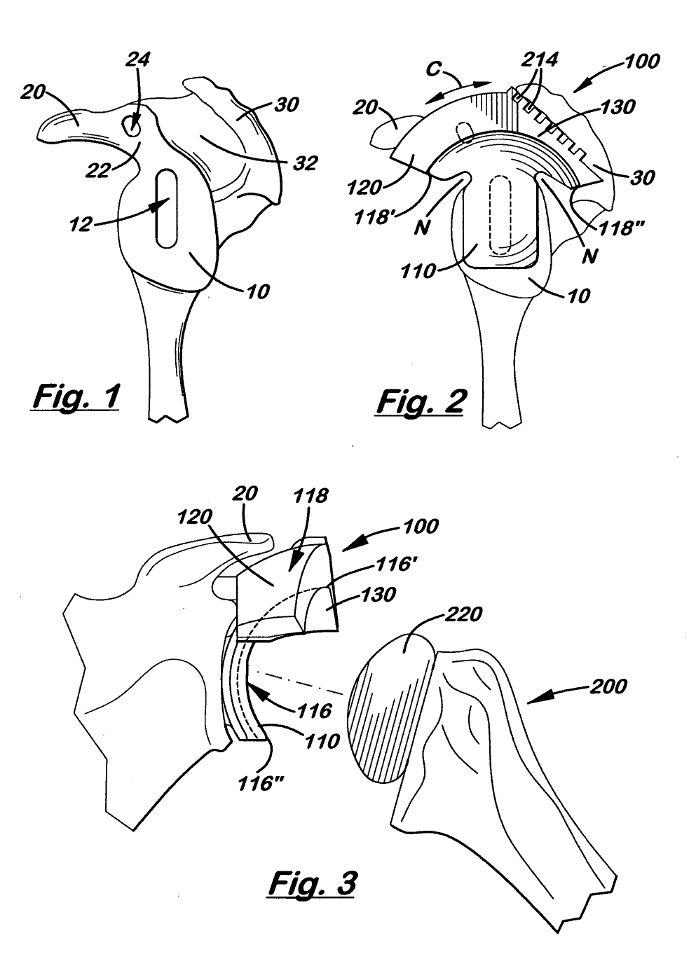

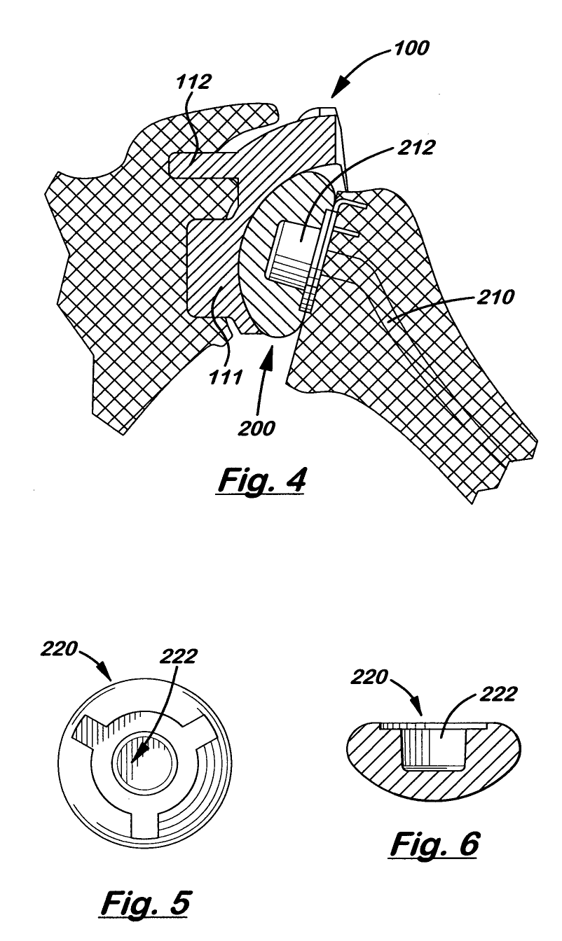

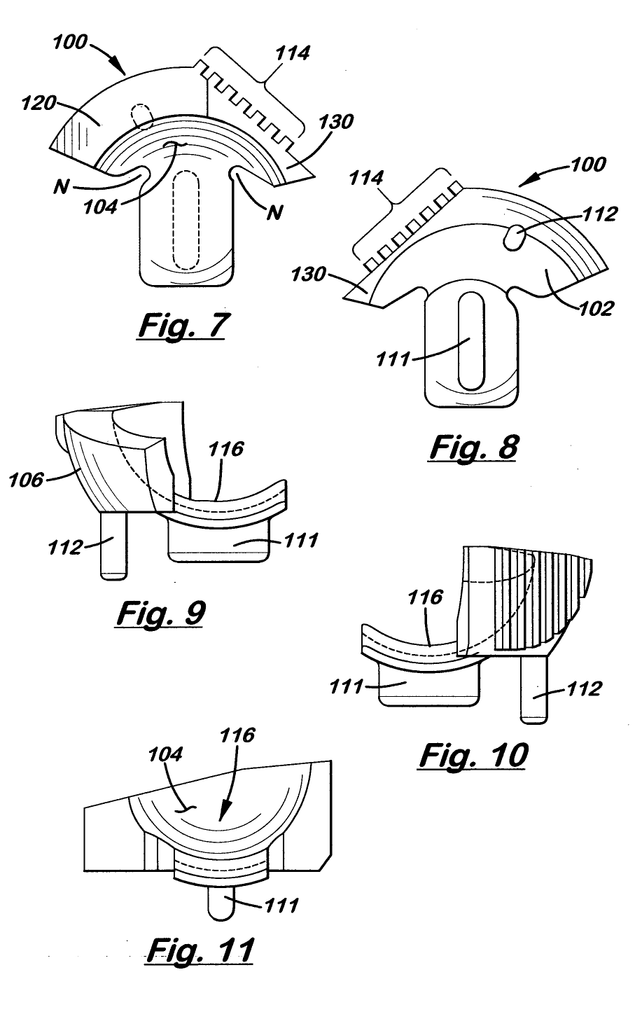

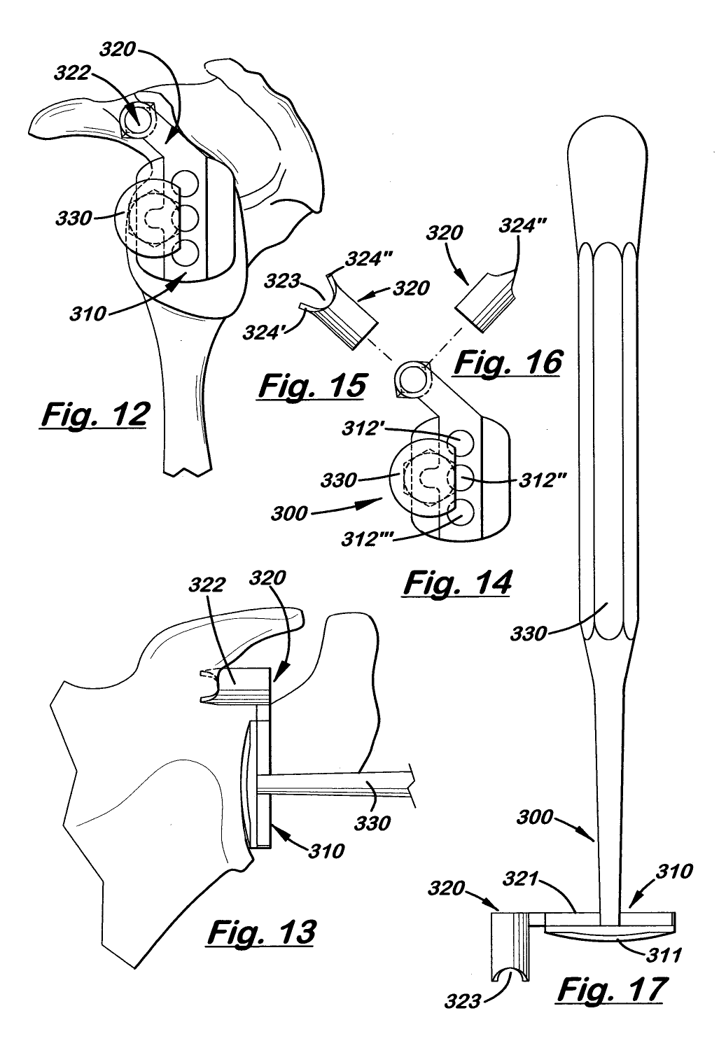

As shown in FIGS. 2-11, the preferred embodiment of the invented glenoid component 100 has a generally cup- or concave-shape main body of less than a hemisphere. The main body has inferior region 110, anterior region 120, and posterior region 130 for securing the glenoid component 100 to the three most lateral projections/extremities of the scapula viz. the glenoid fossa 10, the coracoid process 20, and the acromion process 30 (see FIGS. 1 and 2). The glenoid component 100 may be constructed of highly cross-linked polyethylene, ultra high molecular weight polyethylene, or other rigid biocompatible material(s).

The component 100 may also be described as having superior hood extension 118, which extends anteriorly and posteriorly, to provide stability to the joint, plus an inferior region 110 that extends downward from the hooded extension 118. The inferior-anterior and inferior-posterior edges of the component 100 are preferably curved or notched inward (N in FIGS. 2 and 7) so that the inferior region 110 preferably measures less than 1½inches (more preferably about ¾-1 V₂ inches, and most preferably only about ¾-1 inches), in the anterior-posterior direction. This inferior region 110 that is narrow in its anterior-posterior dimension increases the range of motion of the humeral head 220 within the within the joint, especially in terms of greater arm elevation in the anterior and posterior directions. Thus, one may see that the combination of the constrained hood 118, and the semi-constrained inferior portion of the component 100 resulting from the curved/notched edges (N), result in excellent stability combined with excellent range of motion.

Herein, the glenoid component 100 is described in relationship to the anatomy of the shoulder joint. As shown in FIGS. 7 and 8, the glenoid component comprises a medial surface 102 and a lateral surface 104 when oriented in the gleno-humeral joint. In the preferred embodiment, the medial surface 102 of the glenoid component comprises three points of attachment to the scapula.

Preferably, the inferior-medial attachment 111 is an elongated extension for attachment to the glenoid fossa 10 by means of insertion into a drilled or reamed slot/hole in the glenoid fossa 10. The inferior-medial or glenoid attachment 111 may be keel shaped, pin shaped, or another elongated shaped protrusion, so long as it prevents the glenoid component 100 from pivoting in the glenoid fossa 10. The glenoid attachment 111 may include a hole for helping to secure the attachment with cement in the glenoid fossa 10. Most preferably, the attachment is keel shaped, as its elongated plate-like shape helps prevent rotation in the glenoid fossa 10.

The attachment 112 on the anterior-medial side of the glenoid component 100 is preferably a peg or post for securing the glenoid component 100 to the base 22 of the coracoid process 20, by means of insertion into a drilled/reamed hole in the coracoid process 20. Preferably, the coracoid attachment 112 has a length that is adapted to not puncture through the coracoid process 20 because the brachial plexus and the brachial artery passes on the medial side of the coracoid process 20. Typically, this length is in the range of 0.5-0.75 inches.

The attachment 114 on the posterior-superior side of the glenoid component is preferably a plurality of ridges for securing the glenoid component 100 to the acromion process 30, by means of cement adhering to the ridges 114 and spaces between the ridges 214 (see FIG. 2). The acromial ridges 114 may be oriented in any direction on the superior surface 106 of the glenoid component 100 (see FIG. 2), but most preferably are oriented perpendicular to the circumference C of the hood extension 118.

As shown to best advantage in FIGS. 3 and 11, the lateral surface 104 of the glenoid component 100 comprises a concave interior curvature 116 for articulating with the humeral component 200 and hood extension 118 for preventing the humerus from translating in the superior direction. Preferably, the interior curvature 116 is adapted to articulate against the humeral head 220 of the humeral component 200 (see FIGS. 3 and 4). This curvature 116 is structured to optimize the patient's range of motion while providing superior constraint via hood extension 118. The curvature 116 preferably extends in the range of about 120-160 degrees from its superior extremity 116′ to its inferior extremity 116″ (see FIG. 3). The inventor envisions that various sized glenoid components would be constructed and, during a surgery, the surgeon would implant the glenoid component that is best fitted to the patient's joint. The hood extension 118 may be extended to prevent the humeral head 220 from translating in the superior direction. For example, in the preferred embodiment, the hood extension 118 has a curvature C in the anterior and posterior directions (from its anterior extremity 118′ to its posterior extremity 118″) in the range of about 100-140, and more preferably 110-130 degrees (see FIG. 2), and preferably has a radius in the range of about ¾-1¼ inches, depending mainly on the size of glenoid component required for the patient.

Herein, the humeral component 200 is described in relationship to the anatomy of the humerus. The humeral component 200 preferably comprises a stem portion 210 and a head portion 220 (see FIGS. 3 and 4). Preferably, the stem 210 is an elongated member having a distal end and a proximal end. The distal end of the stem 210 is implanted in the medullary canal of the humerus, as shown in FIG. 4. The proximal end of the stem comprises a post 212. Preferably, the post 212 extends beyond the proximal surface of the humerus. The head portion 220 of the humeral component 200 is preferably spherical in shape and comprises a detent 222 on its inferior surface for connecting the head 220 to the post 212 on the stem 210 (see FIGS. 4-6). Preferably, the head 220 is removably connected to the post 212 via a friction fit, but other attachment methods may be used such as a lock and key system. Preferably the post 212 and detent 222 system is modular in design, so that various sized heads 220 may be fitted onto the stem 210 in order to determine the appropriate replacement head 220 for a particular patient.

The preferred method of implantation comprises the Cofield anterior deltopectoral extensile approach, with the following preferred adaptations, and with an invented jig 300, being used to ensure that drilling into the bone is done at the proper locations.

Prior to surgery, X-rays are taken of the patient's shoulder. The X-rays have markers or indicators to help the surgeon approximate the appropriate size glenoid component for the patient. The inventor envisions approximately five sizes of glenoid component (extra-small, small, medium, large, and extra-large); however, other sizes may be constructed as well.

In the preferred method, the patient is placed in the “beach chair” or sitting up position. The first incision is made along the deltopectoral groove. Preferably, this incision from the level of the clavicle to the anterior aspect of the deltoid is extended over the acromial joint by 2 cm. The extension of the incision allows for greater exposure in order to perform an acromioplasty and in order to drill into the coracoid. The preferred extensile approach involves detaching the anterior third of the deltoid from its origin on the outer surface of the clavicle to further increase the exposure of the joint. However, the inventor envisions that other less invasive surgical approaches may be used.

Prior to carrying out the steps necessary for glenoid component placement, the surgeon inspects the coraco-acromial arch. For the placement of the invented glenoid component, the inventor envisions that an anterior acromioplasty will be performed and a distal clavicle excision may be necessary. The inventor also envisions that the surgeon will remove the soft tissue from the subacromial surface to expose cancellous or spongy bone in order for the bone to receive the cement. Further, the cancellous at the base of the coracoid process is exposed using a Rongeur, which uses a sharp tooth to “bite” the bone away.

After the coraco-acromial arch is assessed and the glenoid is exposed, a centering hole is started in the glenoid fossa 10 care of a drill or a small burr. Once the hole is drilled in the glenoid fossa 10, a reamer or burr is positioned in the centering hole and used to smooth and expose the glenoid bone.

After preparation of the coraco-acromial arch, an invented jig is then installed into the glenoid fossa, to ensure proper location of drilling into the glenoid bone and the coracoid process. One embodiment of the jig 300 is shown in FIGS. 12-17. An appropriately-sized jig is selected base on the sizing done by means of the X-ray markers. The jig 300 is constructed generally according to the bone structure, specifically the relationship between the patient's coracoid process and glenoid fossa center, in order to determine where to drill the desired holes into the coracoid process of the scapula. Specifically, the jig 300 mimics a patient's bone structure, in that it comprises a generally round or oval glenoid template 310 with a convex undersurface 311 for resting in the previously-exposed and -prepared glenoid face, and an arm-like coracoid template 320 that extends from the glenoid template 310 at an angle appropriate for extending along the base of the coracoid process. The glenoid template 310 and coracoid template 320 comprise drill guides that mimic the relationship of the attachment points 111 and 112 on the glenoid component 100, so that, after the drill guides of the jig 300 are used to drill into the coracoid process and glenoid fossa, the resulting holes are properly positioned to receive the attachment structure (attachment point 111 and 112) of the glenoid component 100. Preferably, there are five to six sizes of jigs for the surgeon to choose from, and possibly up to eight sizes, for properly matching the size or shape of the patient's bone structure, and distance from the glenoid fossa center to the base of the coracoid process.

In the preferred embodiment, the jig 300 comprises glenoid template 310, coracoid template 320, and handle 330 (see FIGS. 12-17). Preferably, the glenoid template 310 comprises three holes 312′ 312″, and 312′″ to allow for drilling into the glenoid fossa 10. The coracoid template 320 preferably comprises an arm 321 extending from the glenoid template 310 and a coracoid drill hole guide 322, which is a hollow, generally cylindrical portion or wall that extends from the arm 321 generally perpendicularly to the plane of the arm. In the preferred embodiment, the coracoid drill hole guide 322 comprises two prongs 324′ and 324″ that are angled out away from the axis of the guide 322, and that fit on either side, superior and inferior, of the base of the coracoid 22 (see FIGS. 13, 15 and 16). Preferably, the jig 300 comprises no moving parts. The handle 330 is used to steady the jig 300 while the surgeon drills the holes.

The jig 300 is installed so that the glenoid template 310 is positioned over the glenoid face with the hole 312″ of the glenoid template 310 aligned with the centering hole in the glenoid face. In doing so, the coracoid template 320 is also positioned so that prongs 324′ and 324″ of the coracoid template 320 extend on either side of the base of the coracoid process 22, as shown in FIGS. 12 and 13. The hole guide 322 engages the base of the coracoid 22, with the base of the coracoid received in the space 323 between the prongs 324′ and 324″ and with the prongs preventing rotation of the template 320 and the entire jig 300 relative to the bone.

After placement of the jig 300, a second quarter-inch drill with a stop is introduced through jig hole 312″ and into the centering hole in the glenoid fossa and drilled to the appropriate depth. A rubber plug or other metal peg (not shown) is then placed into the hole 312″ and the centering hole in the glenoid face to prevent the jig from moving in the glenoid fossa 10. The rubber plug works in cooperation with the prongs 324′ and 324″ to provide two spaced, temporary anchor points for the jig on the scapula for preventing rotation of the jig 300 relative to the bone, one being on/around the base of the coracoid and one generally in the center of the glenoid face. Thus, the coracoid template 320 and the glenoid template 310 are in a fixed position relative to each other, and the entire jig is in fixed position relative to the bone, to ensure that the surgeon may drill the holes in the proper positions.

After “pegging” of the hole 312″ to the centering hole, two additional quarter-inch holes are made in the glenoid fossa 10 through the superior hole 312′ and the inferior hole 312′″ of the glenoid template 310, resulting in three holes in the glenoid fossa 10.

The surgeon then uses the coracoid drill hole guide 322 to determine where to drill the hole 24 at the base 22 of the coracoid process 20 (see FIG. 1). The coracoid base 22 is preferably drilled with a step drill to create a 6-7 mm hole. Other size holes may be drilled; however, the surgeon must be careful to not drill through the coracoid process 20 because on the medial side of the coracoid process 20 are the brachial plexus and the brachial artery. Following the drilling of the hole in the base of the coracoid 22, the jig 300 and the rubber plug is removed, and the three holes in the glenoid fossa 10 are connected care of a bur to form an elongated hole in the glenoid fossa 10 that will receive the preferred keel-shaped attachment 111.

The final steps of implanting the glenoid component 100 involve placing cement at each of the attachment points. The acromion ridges 114 are trimmed with a saw to match the curvature of the acromion process and adhered to the subacromial space 32 with cement. Sufficient cement is used to fill in the spaces 214 between the ridges 114, in order to form a cement “pad” connecting the ridge region to the acromion process 30. Cement is also placed in the glenoid slot 12 to secure the keel, as well as in the coracoid hole 24 to secure the coracoid attachment 112. The glenoid component 100 is then pressed into place, and excess cement is removed from areas where it is squeezed out from between the glenoid component 100 and the bone.

The jig allows for precise placement of the holes in the bones and for safe drilling of the holes, as well. Further, the drill preferably has a step that stops over-penetration of the drill into the nerves and vessels that are on the other side of the coracoid process.

While the preferred glenoid component attachment structure, preferred methods, and preferred jig have been described with reference to the glenoid component shown in the Figures, alternative glenoid components may also be within the scope of the invention. For example, a glenoid component extending further around the humeral head 220 may be used, such as a glenoid component that does not have curved/notched (N) inferior-anterior, and inferior-posterior edges. Also, embodiments of the invention may include glenoid components in which the attachment structure is not integrally molded or formed with the main body of the glenoid component, for example, posts, pegs, keels, or other protrusions that are connected to the main body by plastic welding techniques, fasteners, or other fixing techniques. Such non-integral attachment structure may be connected to either the lateral surface or the medial surface of the main body of the glenoid component, or to the inferior surface of the main body of the glenoid component, and then extend out past the medial surface toward the scapula. Certainly, integral attachment structures are preferred because they tend to be more durable and unlikely to loosen in the main body of the glenoid component, and also because they do not involve fasteners or other materials on the lateral surface that would interfere with smooth articulation of the humeral head in the curvature 116. Although less preferred, it is envisioned that some of the attachment structure/protrusions may also comprise or consist of screws or other threaded or otherwise gripping members that extend into the bone and adhere to the bone by means other than or in addition to cement.

While the Figures illustrate a glenoid component that is adapted for a left shoulder, one may see that a mirror image component may be used for the right shoulder. Therefore, the description and details above may be applied to embodiments for the right shoulder.

Although this invention has been described above with reference to particular means, materials, and embodiments, it is to be understood that the invention is not limited to these disclosed particulars, but extends instead to all equivalents within the scope of the following claims.

I claim:

1. A glenoid component for a shoulder prosthesis comprising:
a hood extension defining a superior surface for preventing superior translation of a humeral head;
a stem portion depending inferiorly from said hood extension creating an interior curvature for articulating with said humeral head;
wherein the hood extension comprises a first attachment adapted for attachment to the coracoid process; and
wherein the stem portion comprises a second attachment for attachment to the glenoid fossa.

2. A glenoid component according to claim 1, wherein the hood extension curves in an anterior-posterior direction in the range of 100-140 degrees to prevent anterior and posterior instability, and the stem has an anterior-posterior dimension of less than 1½ inches to allow arm elevation in the anterior and posterior directions.

3. A glenoid component according to claim 1, wherein said first attachment is a member protruding from a medial surface of the glenoid component adapted for being implanted into a drilled hole in the coracoid process.

4. A glenoid component according to claim 1, wherein said second attachment is a member protruding from a medial surface of the glenoid component adapted for being implanted into a drilled hole in the glenoid fossa.

5. A glenoid component according to claim 1, wherein said first attachment is a generally cylindrical post.

6. A glenoid component according to claim 1, wherein said second attachment is keel shaped.

7. A glenoid component according to claim 1, comprising a third attachment adapted for attachment to the acromion process.

8. A glenoid component according to claim 7, wherein said third attachment comprises a plurality of ridges running transverse to the circumference of said hood extension.

9. A glenoid component according to claim 1, wherein said interior curvature is less than a hemisphere.

10. A glenoid component according to claim 1, wherein the hood extension has a curvature anteriorly and posteriorly in the range of 110-130 degrees.

11. A glenoid component according to claim 1, wherein said interior curvature extends superiorly and inferiorly in the range of 120-160 degrees.

12. A glenoid component according to claim 1, wherein said first attachment is a generally cylindrical post that extends out from a medial surface of the glenoid component in the range of 0.5-0.75 inches.

13. A glenoid component for a shoulder prosthesis, the glenoid component comprising:
a generally concave main body for placement in a scapula, the main body having an anterior region adapted to extend to near a coracoid process of a scapula, a posterior region adapted to extend to near an acromion process of the scapula, and an inferior region adapted to extend into a glenoid fossa of the scapula;
a first attachment protrusion extending from a medial surface of the anterior region that is adapted to be imbedded into the coracoid process; and
a second attachment protrusion extending from a medial surface of the inferior region that is adapted to be imbedded into the glenoid fossa.

14. A glenoid component according to claim 13, wherein the inferior region has an anterior-posterior dimension of less than 1½ inches to allow arm elevation in the anterior and posterior directions.

15. A glenoid component as in claim 13, wherein said first attachment protrusion extends from the anterior region 0.5-0.75 inches.

16. A glenoid component as in claim 13, wherein said second attachment comprises a keel.

17. A glenoid component as in claim 13, comprising a third attachment protrusion extending from the posterior region comprising a plurality of ridges adapted for being cemented to the acromion process.

18. Apparatus for treating rotator cuff arthropathy, the apparatus comprising the combination of a glenoid component and a jig for drilling into a coracoid process and a glenoid fossa of a scapula so that the scapula receives the glenoid component, the glenoid component comprising:
a main body having a generally concave lateral surface, a medial surface, and a plurality of protrusions extending from the medial surface, the plurality of protrusions comprising a glenoid protrusion extending from an inferior portion of the main body and adapted to be imbedded into the glenoid fossa, and a coracoid protrusion extending from a superior anterior portion of the main body and adapted to be imbedded into the coracoid process; and
the jig adapted for placement on the scapula to guide drilling of holes in the scapula for receiving the glenoid protrusion and the coracoid protrusion, the jig comprising:
a glenoid template having a glenoid drill guide hole located generally centrally in the template for drilling a hole in the glenoid fossa; and
a coracoid template extending from the glenoid template and having a coracoid drill guide hole distanced from the glenoid drill guide hole and adapted for drilling a hole in the coracoid process;
wherein said glenoid protrusion and said coracoid protrusion on the glenoid component are on said medial surface in positions corresponding to the positions of the glenoid drill guide hole and the coracoid drill guide hole on the jig.

19. Apparatus as in claim 18, wherein said coracoid template comprises an arm extending from the glenoid template in a plane generally parallel to the glenoid template, and wherein the coracoid drill guide hole is defined by a generally cylindrical wall extending perpendicularly from the arm.

20. Apparatus as in claim 19, wherein the generally cylindrical wall comprises two prongs spaced apart from each other for extending on each side of a base portion of the coracoid process.

21. Apparatus as in claim 20, wherein said prongs are on opposite sides of the cylindrical wall.

22. Apparatus as in claim 20, wherein the jig further comprises a handle extending perpendicular to the glenoid template for being held during drilling said holes.

23. A jig for use in treatment of rotator cuff arthropathy, the jig being adapted for placement on a scapula to guide drilling into the glenoid fossa and the coracoid process of the scapula, the jig comprising:
a glenoid template having a glenoid drill guide hole located generally centrally in the template for drilling a hole in the glenoid fossa; and
a coracoid template extending from the glenoid template and having a coracoid drill guide hole distanced from the glenoid drill guide hole for drilling a hole in the coracoid process;
a handle extending from the jig generally perpendicular to the glenoid template for extending out of the rotator cuff area so that the jig can be held against the scapula during drilling.

24. A jig as in claim 23, wherein the coracoid template comprises an arm extending from the glenoid template and a guide wall generally perpendicular to the arm, surrounding and defining the coracoid drill guide hole, and having a plurality of prongs protruding from the wall in spaced relationship for abutting on opposite sides of the base of the coracoid process.

25. A jig as in claim 24, further comprising a peg extending from the glenoid drill guide hole for extending into a hole drilled in the glenoid fossa.

26. A jig as in claim 25, wherein the jig is adapted to be immobilized on the scapula by said peg fixing the glenoid template to the glenoid fossa and the prongs abutting on said opposite sides so that the jig does not rotate on the scapula.

27. A method of treating rotator cuff arthropathy, the method comprising:
providing a glenoid component having a plurality of attachment protrusions;
drilling a hole in the base of the coracoid process;
drilling a hole in the glenoid fossa; and
attaching the glenoid component to the scapula by inserting one of said plurality of attachment protrusions in the hole in the coracoid process and inserting one of said plurality attachment protrusions in the hole in the glenoid fossa.

28. A method as in claim 27, wherein the glenoid component comprises a plurality of ridges on a posterior surface, and the method further comprising cementing the ridges to the acomion process of the scapula.

29. A method as in claim 27, further comprising cementing said plurality of attachment protrusions into the bone of the scapula.
